(19) United States
(12) Patent Application Publication
(10) Pub. No.: US 2025/0041624 A1
(43) Pub. Date: Feb. 6, 2025

(54) METHOD FOR ENHANCING ACTIVITY OF PHOTOSENSITIZER BY MAGNETIC FIELD

(71) Applicants: PEKING UNIVERSITY, Beijing (CN); SOUTH CHINA UNIVERSITY OF TECHNOLOGY, Guangzhou (CN)

(72) Inventors: Junlong ZHANG, Beijing (CN); Song GAO, Guangzhou (CN); Zishu YANG, Beijing (CN); Linan ZHOU, Guangzhou (CN); Shaojun ZHANG, Beijing (CN); Bingwu WANG, Beijing (CN)

(73) Assignees: PEKING UNIVERSITY, Beijing (CN); SOUTH CHINA UNIVERSITY OF TECHNOLOGY, Guangzhou (CN)

(21) Appl. No.: 18/719,851

(22) PCT Filed: Dec. 9, 2022
(86) PCT No.: PCT/CN2022/138126

§ 371 Date: Jun. 14, 2024

(30) Foreign Application Priority Data
Dec. 17, 2021 (CN) 202111556554.6

(51) Int. Cl. A61N 5/06

(52) U.S. Cl. CPC A61N 5/062

(57) ABSTRACT

The present invention discloses a method for enhancing the activity of a photosensitizer by a magnetic field, in which the photosensitizer is placed in the magnetic field under light conditions, thus improving the cytotoxicity of the photosensitizer to tumor cells and increasing the activity of the photosensitizer, thereby further enhancing the apoptosis of cancer cells or an inhibitory effect on tumor tissue by using photodynamic treatment methods, which helps to improve an effect of photodynamic therapy.

### TECHNICAL FIELD

The present invention relates to the technical field of application of nonlinear optical photosensitizers, and in particular relates to a method for enhancing the activity of photosensitizers by using a magnetic field.

### BACKGROUND ART

Currently, tumor treatment is mainly by means of surgical resection, radiotherapy and chemotherapy. Conventional means has problems such as being very injurious to the body, easily restricted, and therapy cannot be realized. Photodynamic therapy, as a new approach to tumor therapy, can remedy the deficiencies in existing approaches in some respects, and has good prospect for application in the combined treatment of tumors for controlling tumor and improving life quality of patient(s). Cure by photodynamic therapy is expected for some early stage cancers that are unsuitable for surgery.

Photodynamic therapy is the irradiation of tumor tissue absorbed with a photosensitizer by laser light at a specific wavelength, and the photosensitizer is excited to convert oxygen into molecules such as strongly active singlet oxygen or free radicals, which oxidize with adjacent biological macromolecules to produce cytotoxic effects that cause cancer cells to be damaged, thereby causing cancer cell apoptosis, microvascular damage, and induction of local immunity.

During photodynamic therapy, singlet oxygen, generated by photosensitizers transferring energy to oxygen, has the strongest activity of killing tumor. And thus, improving the oxygen vacancy at the tumor site is an urgent need to overcome for photodynamic tumor treatment, and problems of photodynamic treatment needed to solve is how to enhance the utilization of singlet oxygen, and to improve the therapeutic effect.

### SUMMARY

In order to solve the problems as described above, the present inventors provide a method for enhancing the activity of a photosensitizer by using a magnetic field, thereby enhancing the effect for tumor cells of singlet oxygen generated by excitation of the photosensitizer, by controlling the magnetic field strength condition under laser irradiation, and further enhancing cytotoxicity and inducing apoptosis, thereby completing the present invention.

The present invention provides a method for enhancing the activity of a photosensitizer by a magnetic field, in which the photosensitizer is placed in the magnetic field under light conditions, thus increasing the activity of the photosensitizer.

Preferably, in the method, the oxidation efficiency of singlet oxygen generated by the photosensitizer is enhanced under the action of the magnetic field and light.

The magnetic field strength is from 15 to 700 mT, preferably from 35 to 600 mT, more preferably from 50 to 450 mT.

The method for enhancing the activity of a photosensitizer by using a magnetic field provided by the present invention has the following beneficial effects:
(1) In the present invention, the photosensitizer is placed in a magnetic field with a certain intensity to enhance the action intensity and efficiency of the photosensitizer and increase the oxidation of singlet oxygen. Thus, the cytotoxicity of the photosensitizer to tumor cells can be improved, thereby achieving inhibition effect on tumor tissues or killing them.
(2) In the present invention, a substantial increase in the activity of the photosensitizer is achieved by the additional application of a magnetic field to the photosensitizer, which is easy to realize, has obvious effect, and has no other damage or side effect to the organism, and is easily popularized and applied in practical applications.
(3) It is experimentally demonstrated that the method of the present invention is capable of enhancing the activity of a photosensitizer that generates singlet oxygen under light conditions, so the selectability of the photosensitizer is greatly improved, i.e. the method can be applied in a large range.

### BRIEF DESCRIPTION OF FIGURES

FIG. 1 is a graph showing the trend of mfeᵣ as a function of magnetic field strength in Example 1;

FIG. 2 is a graph showing the absorbance-time plot at 350 nm of the reaction product of photosensitizers RB and KI under irradiation conditions with a magnetic field of 0-14 mT in Example 2;

FIG. 3 is a graph showing the absorbance-time plot at 350 nm of the reaction product of photosensitizers RB and KI under irradiation conditions with a magnetic field of 14-135 mT in Example 2;

FIG. 4 is a graph showing the absorbance-time plot at 350 nm of the reaction product of photosensitizers RB and KI under irradiation conditions with a magnetic field of 135-800 mT in Example 2;

FIG. 5 is a graph showing the trend of mfe_{R}, the rate of change of the reaction rate R, as a function of magnetic field strength in Example 2 wherein RB used as the photosensitizer;

FIG. 6 shows the absorbance-time plot at 350 nm with a magnetic field strength of 100 mT in Example 2;

FIG. 7 is a graph showing the trend of mfe_{R}, the rate of change of the reaction rate R, in a non-magnetic field condition and with a magnetic field strength of 100 mT, respectively, in Example 2;

FIG. 8 is a graph showing the trend of CV, the cell viability, as a function of time at magnetic field strengths between 0 and 800 mT in Example 3;

FIG. 9 is a graph showing the appearance of the number of cells after culture at various magnetic field strengths under treating with RB and PBS buffers, respectively, in Example 3;

FIG. 10 shows the relative cell viability at various magnetic field strengths in Example 3;

FIG. 11 shows fluorescence images of HeLa cells placed under a magnetic field of 0, 250 or 800 mT, respectively, in Example 4;

FIG. 12 is a graph comparing the rate of change in fluorescence intensity of HeLa cells under light and a magnetic field of 0, 250 or 800 mT in Example 4;

FIG. 13 is a two-parameter histogram dot plot showing apoptosis of HeLa cells under a magnetic field of 0, 250 or 800 mT in Example 4.

FIG. 14 is a graph depicting Westernblot analysis of cleaved Caspase-3 proteases, Bax and Bcl-2 in Hela cells placed under a magnetic field of 0, 250 or 800 mT in Example 5;

FIG. 15 is a graph depicting quantitative results from Westernblot analysis of Caspase-3 proteases, Bax and Bcl-2 under different magnetic field conditions in Example 5;

FIG. 16 is a graph showing the relative tumor volume change rate for the (2), (4), (5), (7), (8) groups of mice in Example 6;

FIG. 17 shows the appearances of tumor tissues in the (2), (3), (4), (5), (7), (8) groups in Example 6;

FIG. 18 is a graph showing the rate of change in tumor tissue mass for the (5), (7), (8) groups in Example 6;

FIG. 19 is a graph depicting immunohistochemical (IHC) staining tests for Bax, Bcl-2 and cleaved Caspase-3 of tumor tissue sections in Example 6;

FIG. 20 illustrates a hematoxylin-eosin (H&E) staining test of the major organs of mice in Example 6;

FIG. 21 shows the trend of quantum yield of singlet oxygen (¹O₂) as a function of magnetic field strength in Example 2;

FIG. 22 shows the trend of the lifetime of singlet oxygen (¹O₂) as a function of magnetic field strength in Example 2.

### DETAILED DESCRIPTION OF INVENTION

The present invention will be further described in detail below through the specific embodiments. Through these descriptions, the characteristics and advantages of the present invention will become clearer and more apparent.

The present invention provides a method for enhancing the activity of a photosensitizer by a magnetic field to further enhance the activity of the photosensitizer by controlling the action of an external magnetic field under light conditions, thereby enhancing apoptosis of cancer cells or inhibition of tumor tissue by photodynamic treatment methods, and thereby contributes to enhancing the effect of photodynamic treatment.

The present invention provides a method for enhancing the activity of a photosensitizer by using a magnetic field, wherein, the photosensitizer being subjected to a magnetic field under light conditions, thereby enhancing the activity of the photosensitizer.

Preferably, in the method, the oxidation efficiency of singlet oxygen generated by the photosensitizer is enhanced under the action of the magnetic field and light.

The rate of oxidation of singlet oxygen is increased at a magnetic field strength of 34-355 mT compared to a single light condition; and at a magnetic field strength of 50-220 mT, the oxidation rate of singlet oxygen increases by greater than 20%.

The light wavelength is determined based on the excitation wavelength of the photosensitizer. The photosensitizer is selected from a group capable of energy transitions, and being excited under irradiation conditions and inducing to generate singlet oxygen. Preferably, the photosensitizer is one or several selected from the group consisting of porphyrin compounds and metal complexes thereof, chlorin compounds and metal complexes thereof, bacteriochlorin compounds and metal complexes thereof, phthalocyanine compounds and metal complexes thereof, fluoboric dipyrrole compounds and fluorescein compounds, more preferred are porphyrin compounds, chlorin compounds or fluorescein compounds, such as Rose Bengal (RB, irradiation wavelength 500-570 nm), chlorin e6 (Ce6, irradiation wavelength 630-670 nm), Temoporphine (Temoporfin, irradiation wavelength 520/550/590 or 650 nm), zinc phthalocyanine (ZnPc, irradiation wavelength 600-700 nm), 8-(4-methylphenyl)-1,3,5,7-tetraiodo-borofluodipyrrole (irradiation wavelength 560-590 nm).

The concentration of the photosensitizer is from 0.5 mol/L to 70 mol/L, preferably from 1 mol/L to 50 mol/L, more preferably from 2 mol/L to 30 mol/L.

The magnetic field strength is from 15 to 700 mT, preferably from 35 to 600 mT, more preferably from 50 to 450 mT. In the present invention, by testing the photosensitizer under magnetic field and irradiation conditions, response to iodide ions (I⁻), in vitro cytotoxicity and tumor cells in mice, it have found that when the magnetic field strength is above 700 mT, the magnetic field will inhibit the reaction rate of singlet oxygen with I⁻, promote tumor cell growth in vitro and attenuate tumor cell inhibition in mice. If the magnetic field is too weak, the action of singlet oxygen to I, cytotoxicity in vitro and tumor cells in mice are less effective, and the effect of the magnetic field is not evident. When acting on tumor tissue in vivo in mice, under certain light conditions at a magnetic field strength of 250-700 mT, an inhibitory effect on tumor tissue is achieved, and at a magnetic field strength of 200-300 mT, a killing effect on tumor tissue cells is achieved, when tumor tissue is at a depth of 0.2-5 mm in mice.

Further inhibition of cell growth is demonstrated on Hela cells in vitro when magnetic field strength is 200-500 mT, compared to single light conditions, i.e. the rate of inhibiting the growth of Hela cells under magnetic field conditions is greater than the rate of inhibiting the growth under single light conditions.

The action time of magnetic field and irradiation is from 3 to 20 min, preferably from 3 to 15 min, more preferably from 3 to 10 min. If the action time is too short, the effect of enhancing the activity of the photosensitizer is not noticeable, and if the action time is too long, the effect of magnetic field on the photosensitizer is not further intensified.

The irradiation intensity is from 1 to 200 mW·cm⁻², preferably from 3 to 150 mW·cm⁻², more preferably from 5 to 100 mW·cm⁻². The irradiation intensity is selected depending on the depth of the irradiation location. In a preferred embodiment of the present invention, the irradiation depth is less than 0.2 mm and the irradiation intensity is from 1 to 20 mW·cm⁻², preferably from 4 to 10 mW·cm⁻²; the irradiation depth is 0.2-5 mm and the irradiation intensity is from 50 to 150 mW·cm⁻², preferably from 80 to 120 mW·cm⁻².

In the present invention, by controlling the magnetic field and the intensity of the light, the activity of the photosensitizer can be further enhanced to promote the oxidative response of singlet oxygen to tumor cells or tumor tissue, thereby enhancing cytotoxicity to tumor cells and promoting apoptosis, and enhancing the effect of photodynamic therapy.

### EXAMPLES

### Example 1

(1) The photosensitizer Rose Bengal (RB) (the purity >98%, available from Saen Chemical Technologies (Shanghai) Ltd. Co., branded as Energy Chemical) and singlet oxygen fluorescent probe (SOSG) are dissolved in water (SOSG available from ThermoFisher Scientific (China) Co., Ltd.). In the resulting solution, the photosensitizer and SOSG are both at a concentration of 10 μmol/L. In a magnetic field with certain strength, the solutions are exposed to a 561 nm LED lamp (light emitting diode) at a power of 5 mW·cm⁻² and tested for fluorescence spectra at 0, 15, 30, 45, 60, 90, 120 and 180 seconds of light exposure, respectively, resulting in emission intensity (1525 nm) versus time (t) curves. The above fluorescence spectroscopy test is performed at a magnetic field strength of 0, 10, 35, 85, 180, 300 mT, respectively (external magnetic field provided by a neodymium iron boron permanent magnet).

(2) The photosensitizer chlorin e6 (Ce6, Chlorin e6) (available from Saen Chemical Technologies (Shanghai) Ltd. Co., branded as Energy Chemical) and singlet oxygen fluorescent probe (SOSG) are dissolved in water, both with a concentration of 10 μmol/L in the resulting solutions. In a magnetic field with certain strength, the solutions are exposed to a 640 nm LED lamp (light emitting diode) at a power of 5 mW·cm⁻² and tested for fluorescence spectra at 0, 15, 30, 45, 60, 90, 120 and 180 seconds of light exposure, respectively, resulting in emission intensity (1525 nm) versus time (t) curves. The above fluorescence spectra are tested at a magnetic field strength of 0, 10, 30, 70, 150, 250 mT, respectively.

The external magnetic field is supplied by Neodymium Iron Boron Permanent Magnet. Fluorescence spectra are tested using Edinburg FLS980 Steady State Transient Fluorescence Spectrometer with a detector of PMT R928 and a light source of 450 W ozone-free xenon lamp with excitation wavelength of λₑₓ=488 nm.

Under irradiation conditions, photosensitizer is excited to produce singlet oxygen ¹O₂, and fluorescent probe SOSG reacts with ¹O₂ to give endoperoxide with green fluorescence emission characteristic, with emission wavelength of 525 nm under excitation wavelength conditions of 488 nm. The reaction has a reaction rate of r.

In the present invention, through application of light and a magnetic field, the magnetic field effects (MFEs) is quantified by mfe_{R}, the change rate of the reaction rate r, mfe_{R}=(r_{B}−r₀)/r₀×100%, where r₀ is the reaction rate at a magnetic field strength of 0 mT and r_{B} is the reaction rate at a magnetic field strength of B mT. Where the reaction rate (r) is proportional to the slope of the tangent of a curve (first derivative of the curve) of the emission intensity (I_{525 nm}) versus time (t). Under magnetic field strengths of 0, 10, 35, 85, 180, 300 mT and from the time of starting to illuminate and place in magnetic field (the time recorded as 0), the first derivative of the I_{525 nm}−t curve is 1, 1.05, 1.09, 0.99, 1.12, 1.14 (normalized).

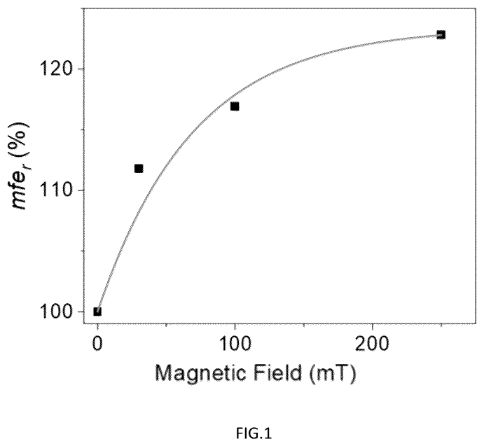

By above tests, curves of mfeᵣ versus magnetic field strength B are obtained with RB as the photosensitizer, as shown in FIG. 1 in particular.

As can be seen in FIG. 1, r increases as applied magnetic field increases from 0 mT to 250 mT and peaks at magnetic field strength of 250 mT, at which mfeᵣ is 25%, indicating that applying magnetic field with certain strength can increase reaction rate of SOSG with ¹O₂.

With Ce6 is used as the photosensitizer, variation of mfeᵣ with magnetic field strength B is similar to that with RB used as the photosensitizer, and the first derivative of I_{525 nm}−t curve at time 0 is 1, 1.09, 1.33, 1.23, 1.16, 1.12 (normalized) at 0, 10, 30, 70, 150, 250 mT.

### Example 2

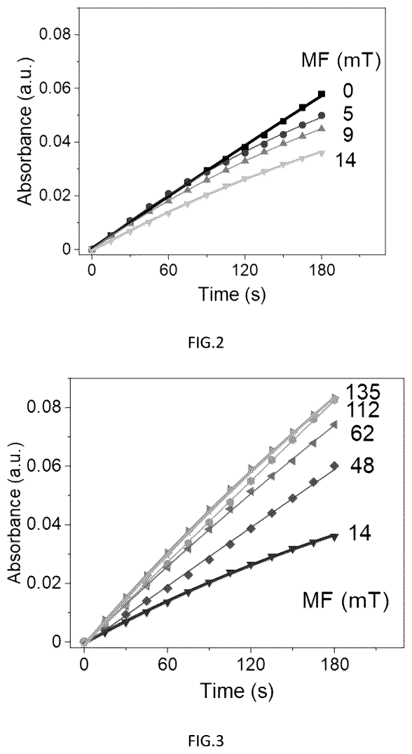

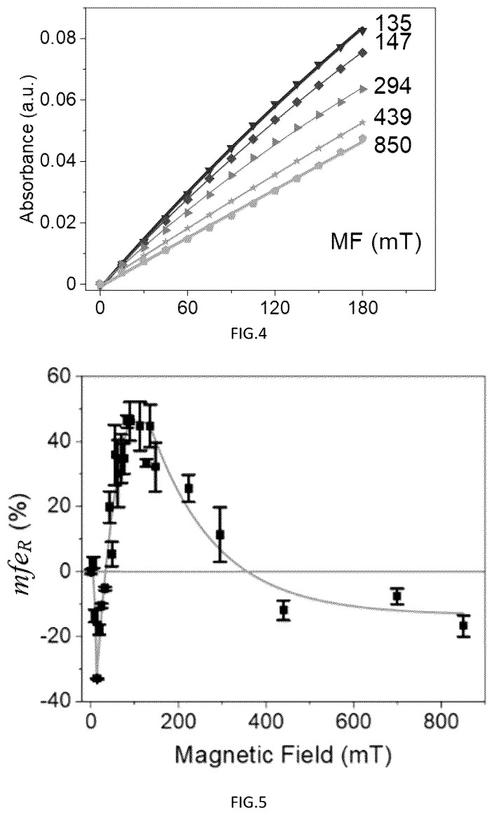

(1) The photosensitizers Rose Bengal (RB) and potassium iodide KI are dissolved in water, with the concentrations of photosensitizer and KI is 10 μmol/L and 10 mmol/L, respectively in the resulting solution. In a magnetic field with certain strength, the solutions are exposed to a 561 nm LED lamp (light emitting diode) at a power of 5 mW·cm⁻² and the absorption spectra are tested every 15 seconds over 5 min and the absorbance at 350 nm is recorded to give absorbance-time curves, as shown in FIG. 2, FIG. 3 and FIG. 4. The above UV-vis absorption spectra tests are performed in the strength range of the magnetic field from 0 to 850 mT respectively.

(2) The photosensitizer chlorin e6 (Ce6, Chlorin e6) and potassium iodide KI are dissolved in water, with the concentrations of the photosensitizer and KI of 10 μmol/L and 10 mmol/L in the resulting solutions. In a magnetic field with certain strength, the solutions are exposed to a 635 nm LED lamp (light emitting diode) at a power of 5 mW·cm⁻², and tested for absorption spectra every 15 seconds over 5 min, and the absorbance at 350 nm is recorded to obtain absorbance versus time curves. The above UV-vis absorption spectra are obtained at a magnetic field strength range from 0 to 850 mT, respectively.

External magnetic field is supplied by East Transformer (Beijing) EM4 electromagnets. Ultraviolet-visible absorption spectra are tested with Agilent 8453 UV/Visible spectrometer equipped with Agilent 89090A thermostat (±0.1° C.).

Under irradiation conditions, photosensitizer is excited to produce singlet oxygen ¹O₂, and KI reacts with ¹O₂ to give I₂ and target detector, iodine triion I₃⁻, characterized by absorption bands centered at 300 and 350 nm in UV-vis absorption spectrum. The reaction rate is R, which is proportional to the slope of absorbance-time curve of iodine triion I₃⁻ at 350 nm.

In the present invention, the magnetic field effects (MFEs) is quantified by mfe_{R}, the change rate of the reaction rate R, mfe_{R}=(R_{B}−R₀)/R₀×100%, where R₀ is the reaction rate at a magnetic field strength of 0 mT and R_{B} is the reaction rate at a magnetic field strength of B mT.

As the magnetic field strength increases, the trend of the change rate mfe_{R} of the reaction rate R when RB is used as the photosensitizer is shown in FIG. 2, FIG. 3, FIG. 4. As can be seen from FIG. 2, FIG. 3, FIG. 4, when the magnetic field strength (MF) increases from 0 mT to 14 mT, mfe_{R} decreases first, with a minimum of 33% at MF=14 mT; thereafter the increase starts, reaching a maximum of 46% in the range of MF=80-135 mT. As MF continues to increase from 135 mT to 850 mT, mfe_{R} rapidly decreases. I.e., under magnetic field conditions of MF=80-135 mT, the change rate mfe_{R} reaches the maximum, the reaction rate is the highest and the magnetic field effect of the reaction is in the “on” state. The trend of mfe_{R} (%) as a function of magnetic field strength B is specifically shown in FIG. 5, where mfe_{R} R>0 when the magnetic field strength is from 34 to 355 mT; and mfe_{R}>20 when the magnetic field strength is from 50 to 220 mT.

With Ce6 is used as the photosensitizer, variation of mfe_{R} of reaction rate R with magnetic field strength B is similar to that with RB used as the photosensitizer, indicating that the kind of photosensitizer has little influence on the reaction of KI with ¹O₂.

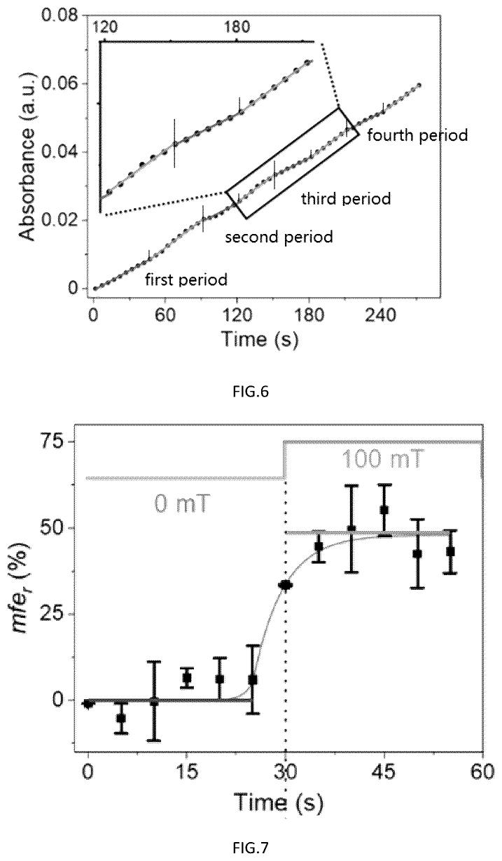

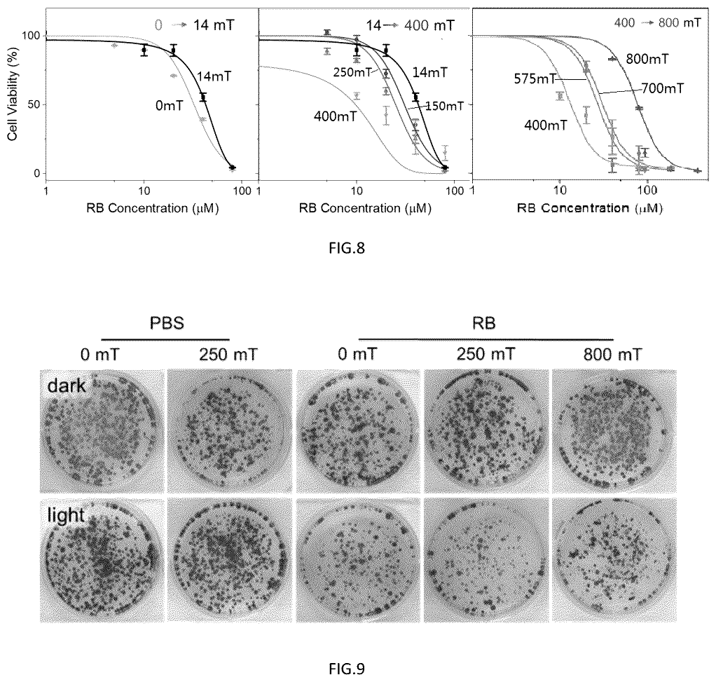

(3) The RB/KI solution in experiment (1) of this Example is placed in a magnetic field modulated with square wave (90 s for the first period, a magnetic field strength of 0 mT for the first 45 s and 100 mT for the last 45 s; 60 s for the last three cycles, a magnetic field strength of 0 mT for the first 30 s and 100 mT for the last 30 s). Under 561 nm LED (Light Emitting Diode) irradiation with 5 mW·cm⁻², absorption spectra are tested every 5 seconds over 5 min. and the absorbance at 350 nm is recorded, to obtain an absorbance-time curve, as shown in FIG. 6. Increased MF increases the R-value significantly suddenly. What can be seen from FIG. 6 that the first derivative (approximated as the slope) of the first half of the period of the absorbance-time curve is significantly smaller than the second half of the period, that is, the R value in the first half of the period is significantly less than that in the second half of the period, and product accumulated and absorbance appear to continue to rise. The R values are calculated for each time at 0 mT and 100 mT, respectively, and as shown in FIG. 7, R value increases by about 45% on average at a magnetic field with 100 mT compared to 0 mT.

(4) Singlet oxygen (¹O₂), quantum yield (ΦΔ) and lifetime (τΔ) are measured by the following experiment. The phosphorescence of singlet oxygen (¹O₂) generated at 1270 nm is determined in air saturated D₂O solution of RB (10 μmol/L). The solution is excited at 561 nm.

The magnetic field effect MFs (Φ_{Δ,B}) is calculated by the following formula: Φ_{Δ,B}=Φ_{Δ,0}×(I_{B}/I₀), where I_{B} and I₀ are peak areas of emission peaks at a magnetic field strength of B and without magnetic field strength, respectively.

The change rate of the magnetic field effect is:

$${mfe}_{Φ} = ( {Φ}_{Δ , B} - {Φ}_{Δ , 0} ) / {Φ}_{Δ , 0} \times 100 \% = ( {I}_{B} - {I}_{0} ) / {I}_{0} \times 100 \% ;$$

The lifetime (τΔ) of singlet oxygen (¹O₂) is affected by the magnetic field effect is as follows:

$${mfe}_{τ Δ} = ( {τ}_{Δ , B} - {τ}_{Δ , 0} ) / {τ}_{Δ , 0} \times 100 \%$$

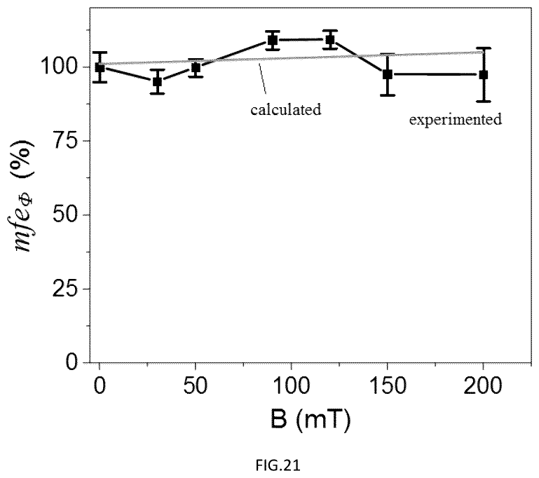

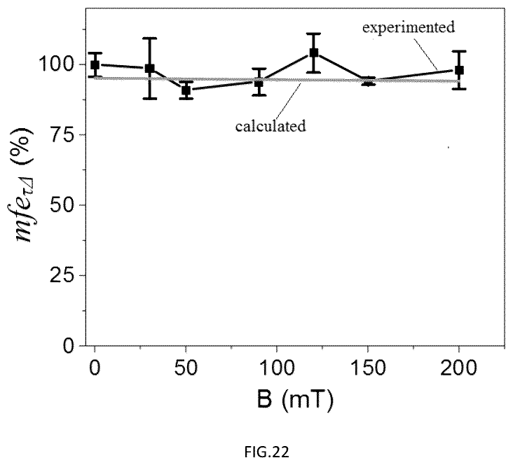

Quantum yield ΦΔ for singlet oxygen (¹O₂) show that, with the magnetic field strength increasing, luminescence quantum yield of ¹O₂ is little influenced by the magnetic field (rate of change is from −5% to +10%), as shown in particular in FIG. 21. By experiment, it proves that its change is not significant, and the fluctuation error is considered to be an effect of systematic errors such as test process differences. On the other hand, the test data of lifetime τΔ for singlet oxygen (¹O₂) is shown in Table 1, and the change trend of mfe_{τΔ} calculated for a magnetic field of 0 mT as 100% is shown in FIG. 22, which shows that the lifetime TA of singlet oxygen (¹O₂) does not change much over the strength range of experimental magnetic field. Experimental results show that the fluorescence of singlet oxygen (¹O₂) of RB does not change much regardless of external field, indicating that the excited state of the photosensitizer is not affected by the magnetic field. Thus, magnetic field strength has a large effect on the reaction rate of ¹O₂ and iodide ions.

TABLE 1

| MF (mT) | τ_{Δ} (μs) |
|---|---|
| 0 | 66.7 ± 2.8 |
| 30 | 65.8 ± 7.1 |
| 50 | 60.7 ± 2.0 |
| 90 | 62.6 ± 3.2 |
| 120 | 69.5 ± 4.7 |
| 150 | 62.8 ± 0.8 |
| 200 | 65.4 ± 4.5 |

### Example 3

(1) Appropriate amounts of HeLa cells are added to DMEM medium (Corning, USA, high glycoform, with glucose concentration less than 4.5 g/L), and then appropriate amounts of 10 wt % fetal bovine serum (FBS), 1 wt % penicillin and streptomycin are added. Cells are cultured at 37° C. in humidified air with 5% (volume fraction) CO₂ for 2 days.

The above Hela cells (2×10³/well) are seeded in 96-well plates for 24 h without light. 100 μL medium and 100 μL aqueous solution of RB with concentrations of 80, 40, 20, 10, 5 μmol/L or 100 μL 0.01 mol/L PBS buffer (phosphate buffered saline) are added respectively, and treated for 24 h, thereafter the cells are washed 3 times with PBS buffer.

DMEM (comprising FBS) medium is added to the 96-well plates for 24 hours in the absence of light. An additional 10 μL of Cell Counting Kit-8 (CCK-8 kit) (Beyotime Biotechnology Co. Ltd.) and 90 μL of DMEM are added to each well, and the light and magnetic field strength are controlled for a subsequent incubation of 30 minutes. The absorbance at 450 nm is read using a microplate reader. Cell Viability (CV) of Hela cells is calculated using the following equation:

CV=(Aₛ−A_{b})/(A_{c}−A_{b})×100%

Where Aₛ is the absorbance of HeLa cells comprising photosensitizer (PS), A_{c} is the absorbance of Hela cells without PS and A_{b} is the absorbance of those without PS and HeLa cells.

The change rate of half inhibitory concentration, mfe_{P} (Cell Viability (%)) is obtained as follows: mfe_{P}=(IC_{50,0}−IC_{50,B})/IC_{50,B}×100%, where IC_{50,0} is half inhibitory concentration at magnetic field strength of 0 mT and IC_{50,B} is half inhibitory concentration at magnetic field strength of B.

Under white light with lighting conditions of 400-700 nm, the power of 5 mW·cm⁻², test is carried out in a magnetic field with strengths of 0-800 mT, and the irradiation and the magnetic field perform for 10 min. The results of the test calculations for mfe_{P} (Cell Viability (%)) at different concentrations of RB and each magnetic field strength are shown in FIG. 8. The RB half inhibitory concentration μmol/L at magnetic field strength of B calculated by logistic regression model fitting according to FIG. 8 are shown in Table 2 below.

TABLE 2

| RB Half inhibitory | magnetic field | |
|---|---|---|
| concentration | strength | mfe_{P} (Cell |
| μmol/L | (mT) | Viability(%) |
| 34.2 ± 1.7 | 0 | — |
| 41.6 ± 1.4 | 14 | −21.5 |
| 31.5 ± 1.0 | 40 | 8.1 |
| 29.7 ± 0.7 | 90 | 13.3 |
| 29.2 ± 0.6 | 150 | 14.8 |
| 26.6 ± 0.3 | 250 | 22.4 |
| 26.1 ± 0.8 | 400 | 23.9 |
| 26.7 ± 1.9 | 575 | 22.1 |
| 38.0 ± 1.1 | 700 | −11.1 |
| 54.1 ± 0.8 | 800 | −58.0 |

As can be seen in FIG. 8, compared to the non-magnetic field condition, the half inhibitory concentration of the photosensitizer at 14 mT on cell proliferation is higher than that under non-magnetic field condition, i.e. the cytotoxicity is reduced (mfe_{P}=−21%). When a magnetic field with the strength gradual increased from 14 mT to 400 mT is applied, minimal IC₅₀ and highest cytotoxicity are obtained at 400 mT, and mfe_{P} is −24%. As the strength of applied magnetic field is gradually increased from 400 to 800 mT, mfe_{P} reaches −58% at 800 mT, which manifested as a promoting effect on cell proliferation. The above results are highly consistent with the change trend of mfe_{P} in Example 1. Thus, the MFE in photocytotoxicity likely arises from the reaction rate of ¹O₂ and biomolecular under the effect of the magnetic field.

In above experiment, with final concentration of photosensitizer of 30 mol/L, CV (%) and mfe (CV) (%) measured and calculated at each magnetic field strength are specifically shown in Table 3, where mfe(CV)=(CV₀−CV_{B})/CV₀×100%.

TABLE 3

| MF(mT) | CV(%) | mfe(CV)(%) |
|---|---|---|
| 0 | 71.1 ± 0.5 | 0 |
| 14 | 89.8 ± 4.2 | −26.3 |
| 150 | 72.5 ± 3.2 | −1.97 |
| 250 | 59.9 ± 2.6 | 15.8 |
| 400 | 42.3 ± 6.7 | 40.5 |
| 575 | 74.1 ± 0.6 | −4.2 |
| 700 | 78.3 ± 3.2 | −10.12658 |
| 800 | 83.0 ± 0.5 | −16.7 |

(2) HeLa cells (1×10³/well) are seeded in 6-well plates, 30 μL aqueous solution of RB (final concentration of 30 μM) or equal volume 0.01M PBS buffer is added as control, and, after 24 h incubation in DMEM (comprising FBS) medium, cells are washed 3 times with PBS buffer.

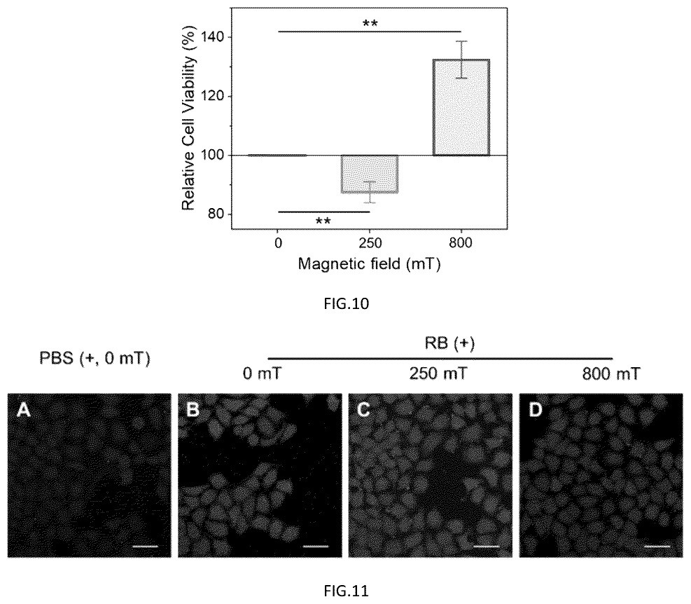

The cells are treated in the dark or with 10 min light (561 nm white light, 5 mW·cm⁻²) under magnetic field conditions at 0 mT, 250 mT, 800 mT respectively. Culture is continued in the dark, and media is changed every other day. After 14 days, it is rinsed with PBS buffer and methanol, and cells are stained with added 0.1 wt % aqueous solution of crystal violet, and the number of cells in the plate is shown in FIG. 9; and the relative cell activity of HeLa cells at a concentration of 30 μmol/L RB under the magnetic field conditions of 0 mT, 250 mT, 800 mT is shown in FIG. 10.

As can be seen in FIG. 9, HeLa cells without co-cultured RB shows no cytotoxicity in the presence and absence of light; RB shows no cytotoxicity against HeLa cells in the absence of light, neither in the presence nor in the absence of a magnetic field; in the presence of photosensitizer RB, cell numbers are significantly lower under light conditions alone relative to PBS-only treatment conditions, is the least under 250 mT conditions, and is more under 800 mT conditions compared to 0 mT and 250 mT conditions and is reduced compared to PBS-only buffer treatment conditions.

As can be seen in FIG. 10, under 250 mT magnetic field conditions, cell viability is inhibited, with cell number of 17% reduction relative to 0 mT; under 800 mT magnetic field conditions, cell viability is enhanced, with cell number of 32% increase relative to 0 mT. *p<0.01, **p<0.05 are considered significant, p is the level of significance at which t-test is performed.

### Example 4

(1) HeLa cells are seeded on sterile glass coverslips and placed in DMEM (comprising FBS) plates for 12 h. Then appropriate amount of 30 μmol/L RB or 0.01 mol/L PBS buffer is added, respectively. After 24 h in dark, H₂DCFDA (dichlorofluorescein diacetate, ≥97%, purchased from Sigma-Aldrich) is added to give final concentration of 10 μmol/L and cells are incubated for another 30 min, and then are washed 3 times with PBS buffer.

Said HeLa cells are placed under the magnetic field conditions of 0 mT, 250 mT, 800 mT, respectively, without irradiation or with 10 min light (561 nm, 5 mW·cm⁻²). Fluorescence images are obtained using Nikon A1R-si laser scanning confocal microscopy (488 nm excitation, 515±15 nm received fluorescence), where H₂DCFDA is the indicator that reacts with cellular singlet oxygen ¹O₂ to increase fluorescence emission intensity (excitation wavelength is 504 nm, and emission wavelength is 529 nm).

Fluorescence images are shown in FIG. 11 (scale represents 25 μm). As can be seen in the Figure, in the absence of magnetic field, the fluorescence intensity of the HeLa cells added with RB is stronger than that with PBS; as for HeLa cells added with RB, under the co-effect of irradiation and magnetic field, the fluorescence intensity thereof under magnetic field of 250 mT is stronger than those under the absence of magnetic field and the magnetic field of 800 mT. Under irradiation for certain time, the amount of generated 102 is close at different magnetic field strengths, and the results shows that the magnetic field strength of 250 mT enhances the oxidation rate in the cells, which is in agreement with the experimental results in Example 1.

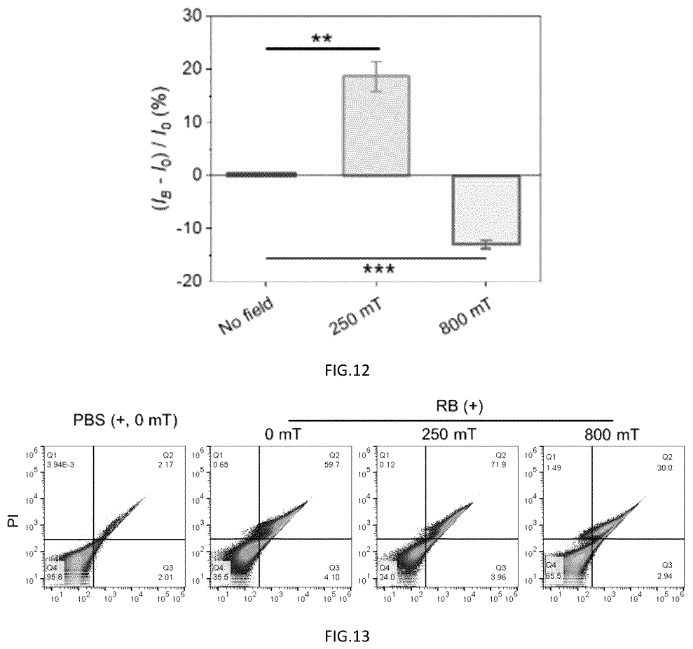

The change rate of fluorescence intensity in Hela cells added with RB under the conditions of light and magnetic field at 0, 250 or 800 mT is shown in FIG. 12. The change rate of fluorescence intensity is (I_{B}−I₀)/I₀×100%, I_{B} is the fluorescence intensity at magnetic field strength of B, and I₀ is the fluorescence intensity without magnetic field. In FIG. 12, ** is the statistical probability p<0.01; *** is p<0.005, and p<0.05 is considered significant.

(2) HeLa cells (2×10⁴/well) are seeded in 6-well plates and cultured for 24 hours, added with RB solution (final concentration 30 μmol/L) or PBS buffer (final concentration 0.01 mol/L), and after treating 24 hours, cells are washed 3 times with PBS buffer.

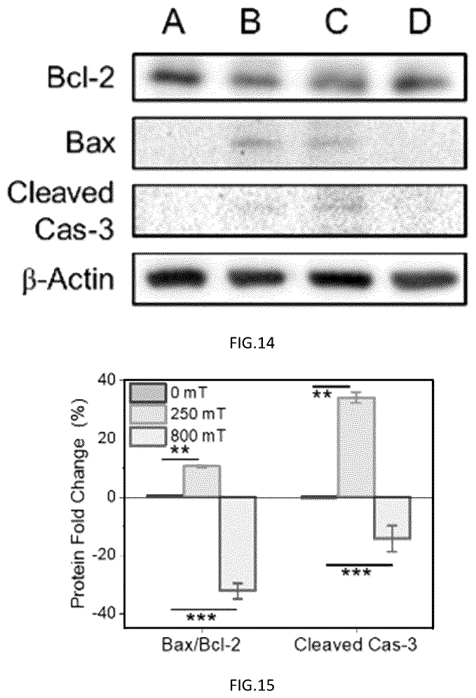

The cells are placed under a magnetic field at 0, 250 or 800 mT, simultaneously with light (561 nm, 5 mW·cm⁻²) or in the dark, and treated 10 minutes. After 24 hours incubation in the dark, cells are stained with Annexin V-FITC/PI Apoptosis Kit (available from Biyotime Biotechnology Co. Ltd), are detected by flow cytometry (BD FACSVerse, Becton Dickinson) and are statistically analysized three times every group, and results are shown in FIG. 13, in which Quadrant Q4 is for the number of healthy cells, Q1 is for the number of early apoptotic cells, Q2 is for the number of late apoptotic cells and Q3 is for the number of necrotic cells.

As can be seen in this Figure, after incubation, the number of healthy HeLa cells added with PBS buffer and without a magnetic field is high; after addition of photosensitizer RB, there is a decrease in healthy cell numbers and an increase in late apoptotic cell numbers with only light and no magnetic field, and 63.8% of apoptotic cells are found; adding a photosensitizer RB, under conditions of applied light and a 250 mT magnetic field, a further reduction in the number of healthy cells, the percentage of apoptotic cells increased to 75.9%, a 19% increase is shown, while the number of healthy cells is significantly increased under the conditions of light and 800 mT magnetic field compared to the conditions of RB without magnetic field, and only 32.9% of cells are apoptotic cells. The results show that singlet oxygen generated after photosensitizer RB is able to suppress the growth of HeLa cells under the conditions of light, and the inhibitory effect is enhanced after the additional application of 250 mT magnetic field; but the additional application of 800 mT magnetic field instead promotes the growth of HeLa cancer cells (less growth compared to PBS buffer without magnetic field). The results shows that exposure to low static magnetic field (250 mT) could promote apoptosis, but high strength magnetic field (800 mT) has the opposite effect. This is consistent with the fluorescence test results in Experiment (1) in this Example.

### Example 5

HeLa cells (2×10⁴/well) are seeded in 6-well plates and cultured for 24 hours, added with RB solution (final concentration 30 μmol/L) or PBS buffer (final concentration 0.01 mol/L), and after treating 24 hours, cells are washed 3 times with PBS buffer.

The cells are placed under a magnetic field at 0, 250 or 800 mT, simultaneously with light (561 nm, 5 mW·cm⁻²) or in the dark. After 24 hours treatment, cells are washed with PBS buffer and collect by centrifugation. Protein is extracted from cells using RIPA lysis buffer (medium lysis strength). Target protein is detected with primary antibody, to identify cleaved Caspase-3 protease, Bax (BCL2-Associated X protein) and Bcl-2 (B lymphocytomatose-2 gene), respectively, with β-Actin antibody as the reference. Images are acquired by Bio-Rad ChemiDoc touch imaging system, and results are shown in FIG. 14, in which, group A is PBS buffer treated HeLa cells (light, no magnetic field); group B is RB treated HeLa cells (light, no magnetic field); group C is RB-treated Hela cells (light, 250 mT magnetic field); group D is RB-treated Hela cells (light, 800 mT magnetic field).

Compared to group B, cleaved Caspase-3 and Bax/Bcl-2 ratios are dramatically increased in group C, while group D decreased. These data indicate that, low static magnetic field (e.g. 250 mT) can promote HeLa apoptosis but high static magnetic field (e.g. 800 mT) induces HeLa cell growth. The change rate of protein expression is (A_{B}−A₀)/A₀×100%, where A_{B} and A₀ are protein expression with and without magnetic field, respectively (obtained by ImageJ software analyzing the bands in FIG. 14). As shown in FIG. 15, the change rates of protein expression at 250 mT and 800 mT are shown in the Table below, where ** is statistical probability p<0.01; *** is p<0.005 and p<0.05 is considered significant.

| magnetic | | |
|---|---|---|
| field | change rate | change rate of |
| strength | of Bax/Bcl-2 | cleaved Caspase-3 |
| (mT) | expression (%) | expression (%) |
| 250 | 10.6 ± 0.5 | 34.1 ± 1.9 |
| 800 | −(32.2 ± 2.7) | −(14.3 ± 4.5) |

### Example 6

Twenty-four five-weeks old female BALB/c nude mice are selected, 1×10⁶ HeLa cells (in 200 μL 0.01 mol/L PBS buffer) are inoculated subcutaneously into the dorsal right flank of each mouse, and a xenograft tumor model is established (Five-weeks old female BALB/c nude mice are obtained from Beijing Agronomy and housed under standard environmental conditions. All animal procedures are approved by Beijing Agronomy Animal Care and Use Committee). After tumors growing up, 25 μL RB (1.0 mg/kg) or an equal volume of PBS buffer at a concentration of 0.01 mol/L are injected intratumorally.

The treatment conditions for dividing the mice into 8 groups (3 mice each) are as follows:
(1) Injection of PBS buffer-no light-no magnetic field (PBS-dark-0mT);
(2) Injection of PBS buffer-light-no magnetic field (PBS-light-0mT);
(3) Injection of PBS buffer-light-250mT magnetic field (PBS-light-250mT);
(4) Injection of PBS Buffer-Light-800mT magnetic field (PBS-light-800mT);
(5) RB-light-no magnetic field (RB-light-0mT) injection (RB-light-0mT);
(6) Injection of RB-light-100mT magnetic field (RB-light-100mT);
(7) Injection of RB-light-250mT magnetic field (RB-light-250mT);
(8) Inject RB-light-800mT magnetic field (RB-light-800mT).

All groups except group (1) receive light 5 min after injection at wavelength of 400-700 nm and power of 100 mW·cm⁻² and light time of 10 min. The tumor area of mice in groups (3), (4) and (6)-(8) are exposed to static magnetic field by using electromagnet during light exposure.

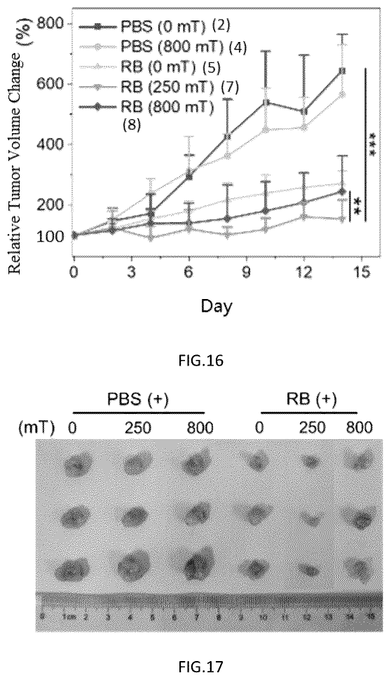

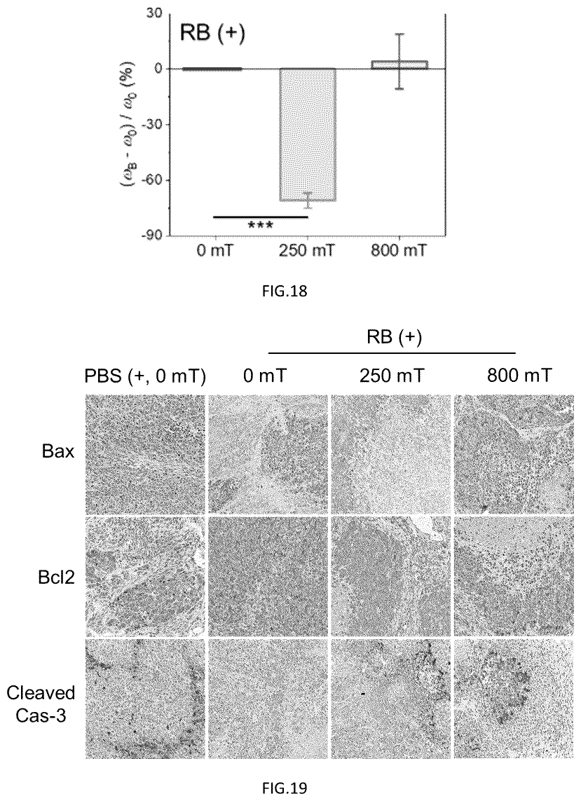

Test (one): Tumor size is monitored with digital calipers every other day for the following 14 days (peripheral measurements of tumors are performed in vitro), and tumors are calculated by Volume=Length×Width×Height=2. Results for groups (2), (4), (5), (7), (8) are shown in FIG. 16, in which, ** is statistical probability p<0.01; *** is p<0.005, p<0.05 is considered significant. Each group is sacrificed 3 mice on day 14 post-injection, tumor tissues are collected for photographing and weighing, and tumor tissues are removed from groups (2), (3), (4), (5), (7), (8) as shown in FIG. 17; the rate of mass change of the tumor tissue in groups (5), (7), (8) is shown in FIG. 18, and the rate of mass change is (ω_{B}−ω₀)/ω₀×100%, where ω_{B} is the tumor mass under magnetic field conditions and ω₀ is the tumor mass in group (5).

From a comparison of measurements of tumor volume and mass in groups (1)-(8), tumor growth is more rapid in the treatment group injected with PBS buffer, i.e., groups (1)˜(4); group (5) (RB-light-0mT) shows inhibition of tumor growth, group (7) (RB-light-250mT) shows further inhibition of tumor growth, and magnetic field effects in group (6) (RB-light-100mT) and (8) (RB-light-800mT) have no significant effect on tumor growth. There appears to be contradiction with in vitro results, presumably due to tumor tissue having comparable volume with limited magnetic pole distance, which results in non-uniform magnetic field distribution in tumor regions and affected by magnetic field effects.

Test (two): After sacrificing the mice on day 14, tumor tissue and major organs (heart, liver, spleen, lungs and kidney) are collected and detected histologically. Tumor tissue and major organs are fixed by 10% neutral formalin solution. Hematoxylin-Eosin (H&E) staining is used to analyze toxicity of light and magnetic field effects on tumor and normal organs. Terminal deoxynucleotidyl transferase-mediated dUTP nick end labeling (TUNEL) staining is used to detect apoptosis in tumor tissue, and expression of cleaved Caspase-3, Bax and Bcl-2 in tumor tissue is simultaneously detected.

FIG. 19 shows immunohistochemical (IHC) staining tests of Bax, Bcl-2 and cleaved Caspase-3 from tumor tissue sections, and it shows significant apoptosis of tumor sections after photodynamic treatment, in which, apoptosis is more pronounced in tumor sections with RB added and a magnetic field of 250 mT, but apoptosis is not significant with RB added with a magnetic field of 800 mT. This indicates expression of the protein in vitro and indicates that exposure to a moderately static magnetic field (250 mT) increased the efficacy of anti-tumor photodynamic treatment in vivo, while the 800 mT magnetic field is reversed. No body weight loss or other abnormal signs are observed in all groups, indicating minimal side effects of RB and magnetic field.

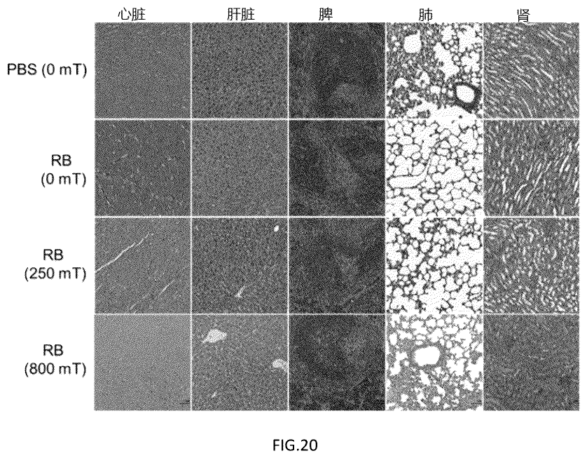

FIG. 20 gives hematoxylin-eosin (H&E) staining tests of major organs, showing that all groups of organs function well, which illustrating that magnetic field effects and photodynamic co-therapy provided in the present invention do not result in systemic toxicity, and contributing to targeted inhibition of tumor tissue.

The invention has been described in detail above with reference to the detailed description and/or exemplary examples and the accompanying drawings, nonetheless, these descriptions are not to be construed as limitations on the present invention. Those skilled in the art understand that Various equivalents substitutions, modifications or improvements may be made to the present subject matter and the embodiments thereof without departing from the spirit and scope of the present invention, all falling within the scope of the present invention. The scope of protection of the present invention shall be subject to the appended claims.

What is claimed is:

1. A method for enhancing the activity of a photosensitizer by a magnetic field, characterized in that, the photosensitizer is placed in the magnetic field under light conditions, thus enhancing the activity of the photosensitizer.

2. The method according to claim 1, characterized in that, the oxidation efficiency of singlet oxygen generated by the photosensitizer is enhanced under the action of the magnetic field and light.

3. The method according to claim 1, characterized in that, the magnetic field strength is from 15 to 700 mT, preferably from 35 to 600 mT, more preferably from 50 to 450 mT.

4. The method according to claim 1, characterized in that, compared to a single light condition, when the magnetic field strength is 34-355 mT, the oxidation rate of singlet oxygen is increased; and when the magnetic field strength is 50-220 mT, the oxidation rate of singlet oxygen increases by more than 20%.

5. The method according to claim 1, characterized in that, the irradiation intensity is from 1 to 200 mW·cm⁻², preferably from 3 to 150 mW·cm⁻², more preferably from 5 to 100 mW·cm⁻².

6. The method according to claim 1, characterized in that, the irradiation depth is less than 0.2 mm and the irradiation intensity is from 1 to 20 mW·cm⁻², preferably from 4 to 10 mW·cm⁻².

7. The method according to claim 1, characterized in that, the irradiation depth is 0.2-5 mm and the irradiation intensity is 50-150 mW·cm⁻², preferably 80-120 mW·cm⁻².

8. The method according to claim 1, characterized in that, the concentration of the photosensitizer is from 0.5 to 70 mol/L, preferably from 1 to 50 mol/L.

9. The method according to claim 1, characterized in that, the concentration of the photosensitizer is from 2 to 30 mol/L.

10. The method according to claim 1, characterized in that, the photosensitizer is selected from a group capable of energy transitions, and being excited under irradiation conditions and inducing to generate singlet oxygen, preferably is one or several selected from porphyrin compounds, chlorin compounds, bacteriochlorin compounds, phthalocyanine compounds, fluoboric dipyrrole compounds and fluorescein compounds, more preferably porphyrin compounds, chlorin compounds or fluorescein compounds.
